(19) United States
(12) Patent Application Publication
(10) Pub. No.: US 2025/0032150 A1
(43) Pub. Date: Jan. 30, 2025

(54) A SYSTEM FOR FORMING A FISTULA

(71) Applicant: CLEARSTREAM TECHNOLOGIES LIMITED, Enniscorthy, County Wexford (IE)

(72) Inventors: John O'Shea, Enniscorthy, Wexford (IE); Sangeetha Rajendraprasad, Enniscorthy, Wexford (IE); Nicola Broaders, Enniscorthy, Wexford (IE)

(21) Appl. No.: 18/696,642

(22) PCT Filed: Oct. 6, 2021
(86) PCT No.: PCT/EP2021/077523

§ 371 Date: Mar. 28, 2024

(51) Int. Cl. A61B 17/34; A61B 17/00

(52) U.S. Cl. CPC A61B 17/3401; A61B 17/00234; A61B 2017/00238; A61B 2017/00305; A61B 2017/00557

(57) ABSTRACT

A catheter system for forming a fistula between two adjacent blood vessels. The catheter system comprises a crossing catheter (110) for introduction into a blood vessel. The crossing catheter has a distal end section and an inflatable balloon (120) disposed in the distal end section for stabilizing the crossing catheter in the blood vessel. The catheter system further comprises a penetration tool (130) for penetrating vessel walls of two adjacent blood vessels to form the fistula. The penetration tool is disposed within the crossing catheter and is movable between a retracted position and an advanced position. In the inflated state, the inflatable balloon (120) comprises at least one blood bypass structure (121) for allowing blood to flow past the inflated balloon.

### TECHNICAL FIELD

The present disclosure relates to a catheter system for forming a fistula between two adjacent blood vessels and a method of forming a fistula using a catheter system.

### BACKGROUND

Peripheral artery disease is a condition caused by abnormal narrowing of a peripheral arteries due to the deposit of atherosclerotic plaque in the walls of the arteries, thereby reducing or blocking blood flow to the limbs. In serious cases, peripheral artery disease can lead to complications such as infection, tissue death leading to amputation or stroke.

Peripheral artery disease can be treated in a number of ways such as by angioplasty or atherectomy. Another treatment option is to place a vascular bypass graft from a location proximal to the blockage to a location distal to the blockage in order to provide an unobstructed path for the blood to circumvent the diseased area. Such vascular bypass grafts are commonly placed in open vascular surgery which are highly invasive procedures which result in long recovery times for the patient.

US 2014/0142677 A1 discloses a system and method for placing a vascular bypass graft in an endovascular procedure, making the procedure less invasive. The system comprises a penetration catheter carrying a penetration tool for penetrating from a peripheral artery to a peripheral vein. The system further comprises a guidewire capture and stabilization catheter which comprises a braid and is designed to capture a guidewire from the penetration catheter in the peripheral vein. The penetration catheter also comprises a stabilizing element in the form of a balloon, expandable braid or malecot.

However, the lumen of the peripheral artery in which the penetration catheter is placed is often heavily calcified and, as a result, uneven, making it difficult for the stabilizing element to stabilize the catheter before puncture of the lumen wall. Furthermore, the use of a balloon stabilizing element blocks blood flow through the vessel during the fistula forming process and increases the risk of thrombosis. Moreover, the system of US 2014/0142677 A1 requires a conventional balloon catheter to dilate the fistula after it has been formed, which greatly increases the treatment time and complexity, as the penetration catheter has to be withdrawn before the conventional balloon catheter can be inserted.

There is hence a need in the art for a new type of catheter system, which provides good stabilization in an uneven, heavily calcified lumen.

There is further a need in the art for a new type of catheter system which allows blood flow through the vessel during the fistula forming procedure to thereby reduce the risk of thrombosis.

There is further a need in the art for a new type of catheter system which reduces the treatment time and makes the procedure simpler by eliminating the need for a separate balloon catheter to expand the fistula.

There is further a need in the art for a new type of catheter system where the balloon surface area has minimum contact with the vessel wall to prevent trauma.

### SUMMARY

In a first aspect of the present disclosure, there is provided a catheter system for forming a fistula between two adjacent blood vessels. The catheter system comprises a crossing catheter for introduction into a blood vessel. The crossing catheter has a distal end section and an inflatable balloon disposed in the distal end section for stabilizing the crossing catheter in the blood vessel. The catheter system further comprises a penetration tool for penetrating vessel walls of two adjacent blood vessels to form the fistula, wherein the penetration tool is disposed within the crossing catheter and is movable between a retracted position and an advanced position. In the inflated state, the inflatable balloon comprises at least one blood bypass structure for allowing blood to flow past the inflated balloon.

In some embodiments, this may result in a catheter system which allows blood flow through the vessel during the fistula forming procedure to thereby reduce the risk of thrombosis.

In some embodiments, this may further result in a catheter system which reduces the treatment time and makes the procedure simpler by eliminating the need for a separate balloon catheter to expand the fistula.

Throughout this disclosure, the term ‘fistula’ is used to denote a connection or passageway.

Throughout this disclosure, the term ‘crossing catheter’ is used to denote a catheter which is suitable for forming a fistula in a blood vessel.

Throughout this disclosure, the term ‘blood bypass structure’ is used to denote a structure which prevents the inflatable balloon from blocking the entire diameter of the blood vessel and thereby allows blood to flow past the inflated balloon.

The blood bypass structure may comprise a spiral channel on an outer surface of the balloon.

In some embodiments, this may provide good stabilization in an uneven, heavily calcified lumen.

The spiral channel may be formed by a wire wrapped in a spiral around the outer surface of the balloon.

The wire may be formed from a metal, preferably Nitinol.

The blood bypass structure may comprise two or more spiral channels on the outer surface of the balloon.

Each spiral channel may be formed by a separate wire wrapped in a spiral around the outer surface of the balloon.

In some embodiments, this may provide better stabilization in an uneven, heavily calcified lumen and allow more blood to flow past the stabilizing element.

The spiral channel or channels may be formed around the longitudinal axis of the inflatable balloon.

The blood bypass structure may comprise a spiral ridge on an outer surface of the balloon.

In some embodiments, this may provide good stabilization in an uneven, heavily calcified lumen.

The blood bypass structure may comprise two or more spiral ridges on the outer surface of the balloon.

In some embodiments, this may provide better stabilization in an uneven, heavily calcified lumen and allow more blood to flow past the stabilizing element.

The spiral ridge or ridges may be formed around the longitudinal axis of the inflatable balloon.

The blood bypass structure may comprise multiple bumps on the surface of the inflatable balloon.

In some embodiments, this may provide good stabilization in an uneven, heavily calcified lumen.

The bumps may extend substantially along the entire length of the balloon.

In some embodiments, this may provide better stabilization in an uneven, heavily calcified lumen and allow more blood to flow past the stabilizing element.

The bumps may extend along 60% to 100%, preferably 80% to 100%, of the entire length of the balloon.

The bumps may have a rounded shape.

The inflatable balloon may have a proximal end and a distal end on either side of a centre portion. In the inflated state, the diameter of the balloon may decrease from the proximal end and the distal end towards the centre portion.

In some embodiments, this may provide better stabilization in an uneven, heavily calcified lumen and allow more blood to flow past the stabilizing element.

The crossing catheter may have a penetration lumen for accommodating the penetration tool.

A distal end of the penetration lumen may end in a penetration port through which the penetration tool can exit the crossing catheter.

The penetration port may be disposed adjacent the inflatable balloon.

In some embodiments, this may result in better stabilization of the penetration tool when forming the fistula.

The penetration port may be disposed distally adjacent the inflatable balloon.

The penetration port may be disposed proximally adjacent the inflatable balloon.

The penetration port may comprise a lateral opening in the crossing catheter.

The penetration port may comprise a longitudinal opening in the crossing catheter.

In some embodiments, this may allow one guidewire to be used for advancing the crossing catheter to the treatment site and translating the crossing catheter through the fistula.

The penetration port may be disposed at a distal end of the crossing catheter.

In some embodiments, this may allow the crossing catheter to more easily translate through the fistula.

In some embodiments, this may further result in a catheter system which reduces the treatment time and makes the procedure simpler by eliminating the need for a separate balloon catheter to expand the fistula.

A distal end of the penetration tool may deflect laterally as the penetration tool is moved distally from the retracted position to the advanced position.

In some embodiments, this may allow the penetration tool to more easily form a fistula in a vessel wall.

The penetration tool may comprise a needle.

The penetration tool may comprise a guidewire lumen for accommodating a guidewire. The guidewire lumen may allow placement of the guidewire through the fistula formed by the penetration tool.

In some embodiments, this may allow quick and simple placement of a guidewire through the fistula.

The crossing catheter may comprise a second guidewire lumen for accommodating a second guidewire.

In some embodiments, this may allow the crossing catheter to be advanced to a predetermined crossing point using a separate guidewire.

The inflatable balloon may be a first inflatable balloon. The crossing catheter may further comprise a second inflatable balloon.

In some embodiments, this may result in better stabilization of the penetration tool when forming the fistula.

The second inflatable balloon, in the inflated state, may comprise at least one blood bypass structure for allowing blood to flow past the inflated balloon.

In some embodiments, this may allow better blood flow past the stabilizing element.

The blood bypass structure may comprise a spiral channel on an outer surface of the balloon.

In some embodiments, this may provide better stabilization in an uneven, heavily calcified lumen.

The blood bypass structure may comprise two or more spiral channels on the outer surface of the balloon.

In some embodiments, this may provide better stabilization in an uneven, heavily calcified lumen and allow more blood to flow past the stabilizing element.

The blood bypass structure may comprise a spiral ridge on an outer surface of the balloon.

In some embodiments, this may provide better stabilization in an uneven, heavily calcified lumen.

The blood bypass structure may comprise two or more spiral ridges on the outer surface of the balloon.

In some embodiments, this may provide better stabilization in an uneven, heavily calcified lumen and allow more blood to flow past the stabilizing element.

The blood bypass structure may comprise multiple bumps on the surface of the inflatable balloon.

In some embodiments, this may provide better stabilization in an uneven, heavily calcified lumen and allow more blood to flow past the stabilizing element.

The bumps may extend substantially along the entire length of the balloon.

The bumps may have a rounded shape.

The second inflatable balloon may be disposed proximal or distal of the first inflatable balloon.

The inflatable balloon may be at least partly made from a woven or spun thread.

In a second aspect of the present disclosure, there is provided a catheter system for forming a fistula between two adjacent blood vessels. The catheter system comprises a crossing catheter for introduction into a blood vessel. The crossing catheter has a distal end section and an inflatable balloon disposed in the distal end section for stabilizing the crossing catheter in the blood vessel. The catheter system further comprises a needle for penetrating vessel walls of two adjacent blood vessels to form the fistula. The needle is disposed within the crossing catheter and is movable between a retracted position and an advanced position. The inflatable balloon is adapted for allowing blood to flow past the inflated balloon.

The inflatable balloon may comprise a spiral channel on an outer surface of the balloon for allowing blood to flow past the inflated balloon.

The spiral channel may be formed by a wire wrapped in a spiral around the outer surface of the balloon.

The inflatable balloon may comprise two or more spiral channels on the outer surface of the balloon.

The inflatable balloon may comprise a spiral ridge on an outer surface of the balloon for allowing blood to flow past the inflated balloon.

The inflatable balloon may comprise two or more spiral ridges on the outer surface of the balloon.

The inflatable balloon may comprise multiple bumps on the outer surface of the balloon.

The bumps may extend substantially along the entire length of the balloon.

The bumps may have a rounded shape.

The inflatable balloon may have a proximal end and a distal end on either side of a centre portion. In the inflated state, the diameter of the balloon may decrease from the proximal end and the distal end towards the centre portion.

The crossing catheter may have a penetration lumen for accommodating the needle. A distal end of the penetration lumen may end in a penetration port through which the needle can exit the crossing catheter.

The penetration port may be disposed at the distal end of the crossing catheter.

The penetration port may comprise a lateral or longitudinal opening in the crossing catheter.

A distal end of the needle may deflect laterally as the needle is moved distally from the retracted position to the advanced position.

In a third aspect of the present disclosure, there is provided a method of forming a fistula between two adjacent blood vessels using a catheter system comprising a crossing catheter having a distal end section and an inflatable balloon disposed in the distal end section, and a penetration tool. The method comprises: introducing the crossing catheter into a blood vessel, advancing the distal end of the crossing catheter to a predetermined point where the fistula is to be formed, stabilizing the crossing catheter by inflating the inflatable balloon. The inflatable balloon comprises at least one blood bypass structure for allowing blood to flow past the inflated balloon. The method further comprises moving the penetration tool from a retracted position and an advanced position to form the fistula.

In some embodiments, this may result in a method of forming a fistula which allows blood flow through the vessel during the fistula forming procedure to thereby reduce the risk of thrombosis.

### BRIEF DESCRIPTION OF THE DRAWINGS

To enable better understanding of the present disclosure, and to show how the same may be carried into effect, reference will now be made, by way of example only, to the accompanying drawings, in which:

FIG. 1 shows a side view of a catheter system comprising a crossing catheter for forming a fistula according to the present disclosure. The inflatable balloon of the crossing catheter is shown in the inflated state.

FIG. 2 shows the catheter system of FIG. 1 with a penetration tool extending from the crossing catheter.

FIG. 3 shows a side view of the catheter system of FIG. 1 where the inflatable balloon is in a deflated state.

FIGS. 4A to 4G illustrate a method of using the catheter system of FIG. 1 to form a fistula between an artery and a vein.

FIG. 5 shows an alternative embodiment of a catheter system and crossing catheter according to the present disclosure. The inflatable balloon of the crossing catheter is shown in the deflated state.

FIG. 6 shows the catheter system and crossing catheter of FIG. 5 with the inflatable balloon in the inflated state.

### DETAILED DESCRIPTION

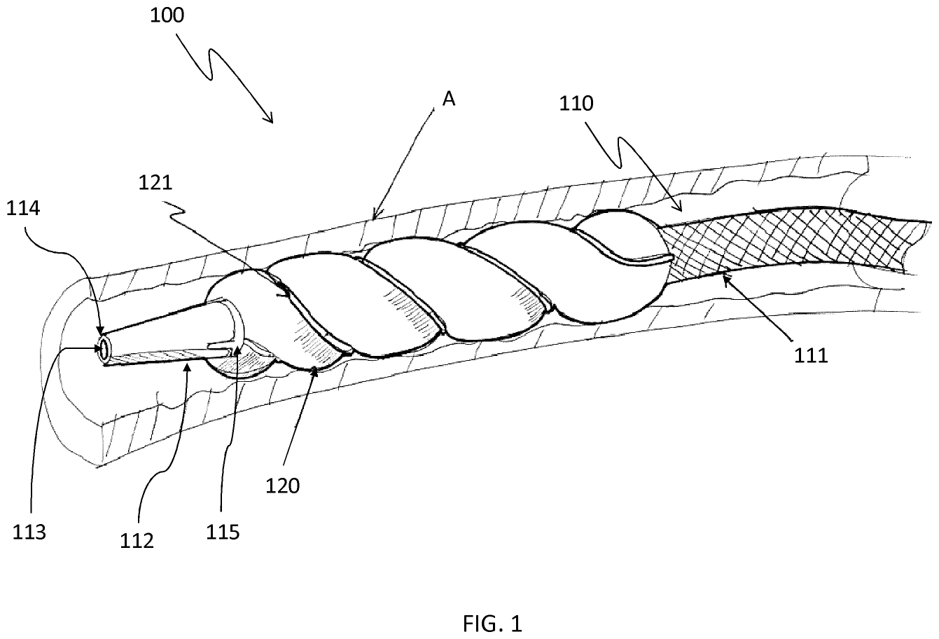

FIG. 1 shows a catheter system 100 for forming a fistula or passage between two adjacent blood vessels. The catheter system comprises a crossing catheter 110 having a shaft 111 and an inflatable balloon 120 disposed at the distal end of the shaft 111. The balloon 120 may be a compliant, semi-compliant or non-compliant balloon and may be formed from materials commonly used in the design of PTA balloons such as, for example, nylon, polyester, Pebax, polyurethane or silicone. The crossing catheter 110 is positioned in an artery A.

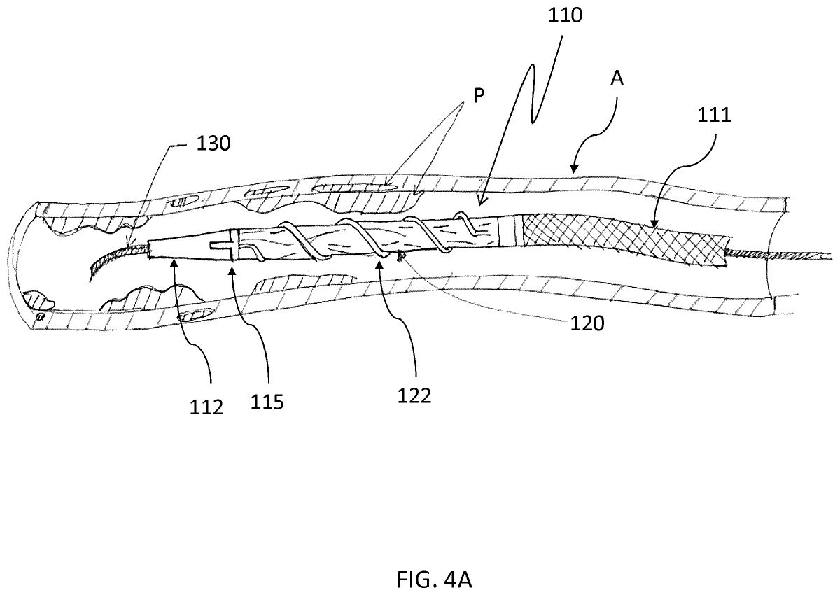

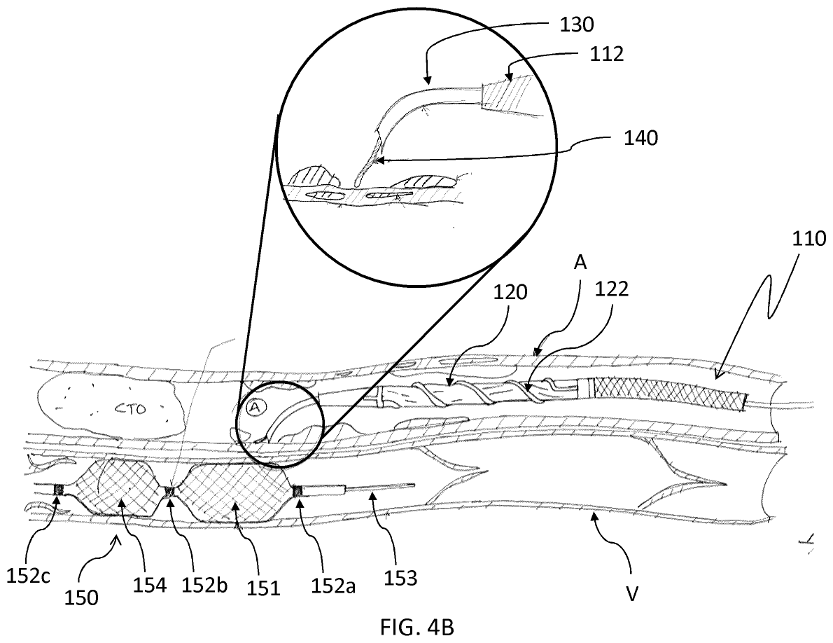

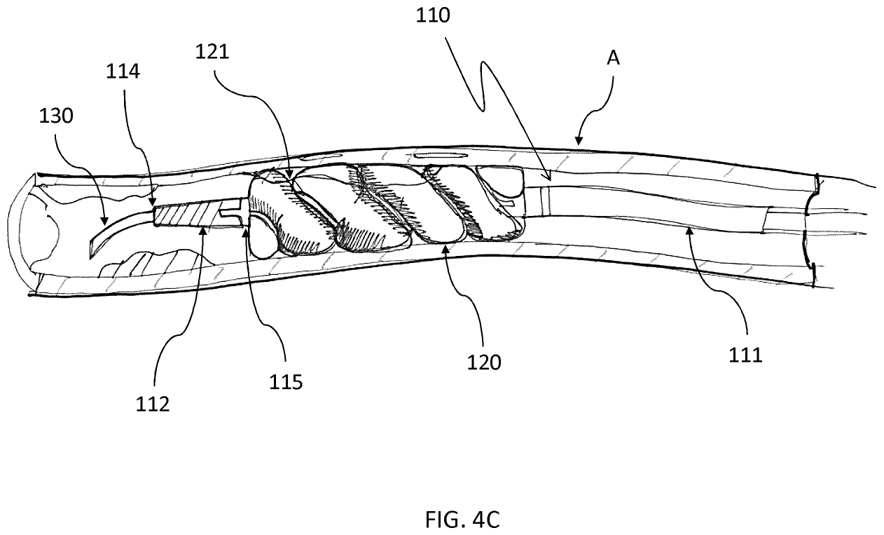

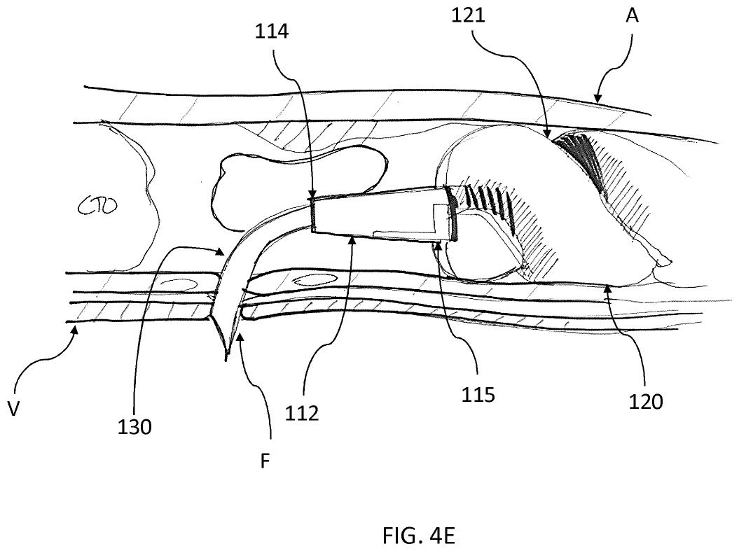

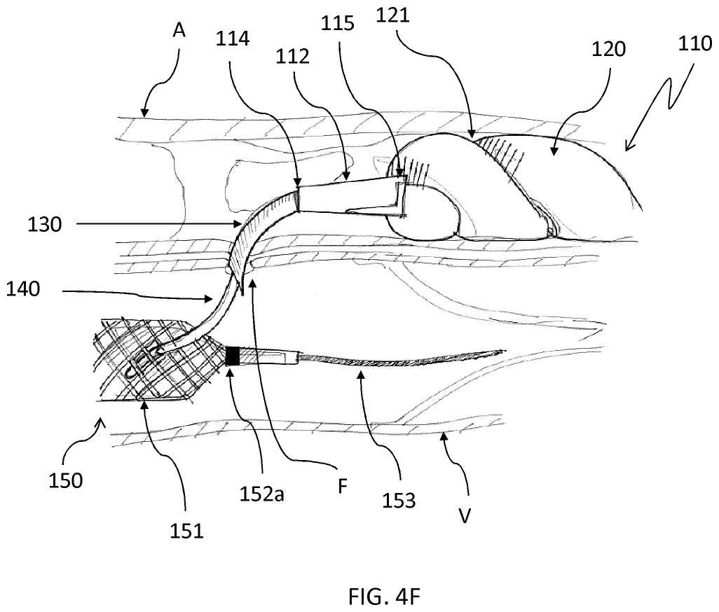

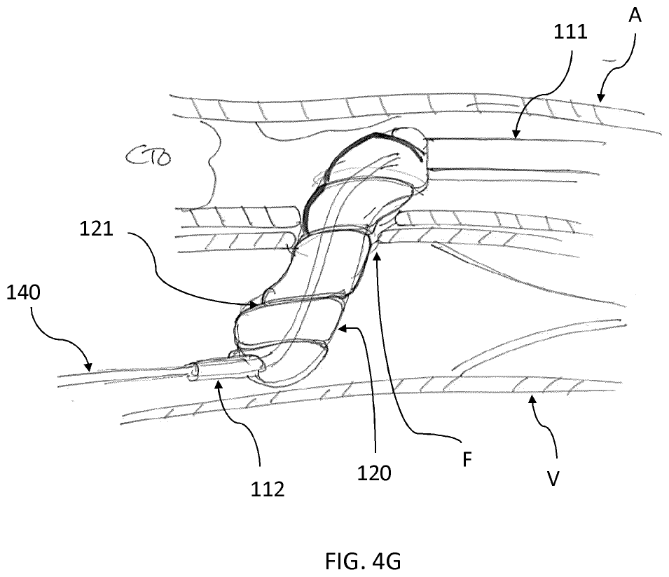

The inflatable balloon 120 stabilises the crossing catheter 110 during formation of the fistula, as is explained in more detail with respect to FIG. 4. The inflatable balloon 120 comprises a blood bypass structure in the form of a spiral channel 121 which extends along the length of the inflatable balloon 120. The spiral channel 121 allows blood to flow past the inflatable balloon 120 when it is in the inflated state and thereby reduces the risk of blood clot formation during formation of the fistula. The spiral channel 121 of the balloon 120 also helps to provide better stabilisation of the crossing catheter 110 in a heavily calcified and uneven lumen, as the spiral channel 121 can better hold on to uneven surfaces in the lumen than a smooth balloon. These fistulas are often formed near a chronic total occlusion where the lumen of the vessel is heavily calcified and uneven. The inflatable balloon 120 can be inflated and deflated through a respective inflation lumen which runs along the shaft 111 of the catheter 110.

The crossing catheter 110 further comprises a distal tip 112 disposed distally of the inflatable balloon 120 and at the distal tip of the crossing catheter 110. Between the inflatable balloon 120 and the distal tip 112, a radiopaque marker 115 is disposed for identifying the location of the distal tip 112 of the crossing catheter 110 under fluoroscopy.

The distal tip 112 is tapered and ends in an atraumatic penetration port 114 through which a penetration tool 130 can exit and extend from the crossing catheter 110, in order to puncture the wall of the blood vessel and form a fistula. The penetration tool 130 is disposed within a penetration lumen 113 within the crossing catheter 120 and can slide within the penetration lumen 113 between a retracted position and an advanced position. A physician can externally manipulate the position of the penetration tool 130, for example, through a pull or push wire mechanism or a rack and pinion mechanism.

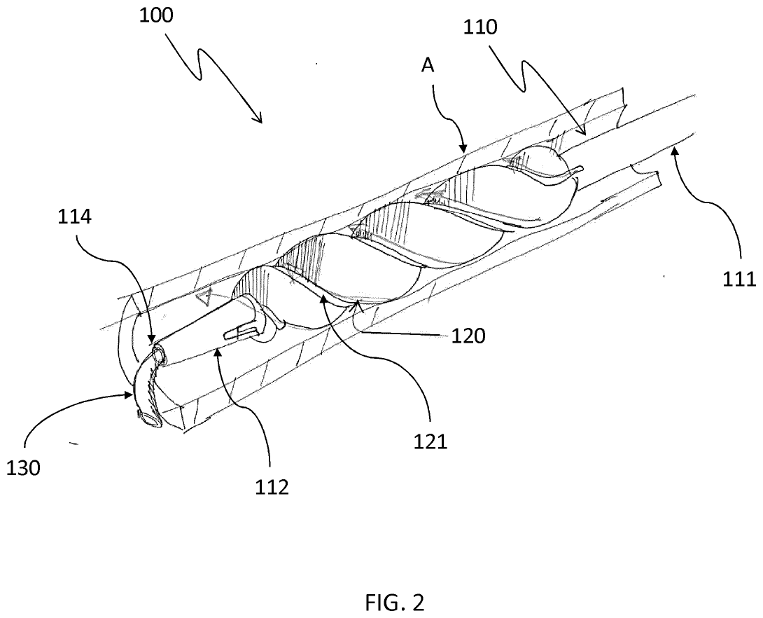

FIG. 2 shows the penetration tool 130 extending from the penetration port 114 in an advanced position. The penetration tool 130 may be a curved, resilient needle with a sharp distal tip which deploys over a radially out-ward curved path as it is advanced to penetrate the vessel wall. In other words, the penetration tool 130 deflects laterally as the penetration tool 130 is moved distally from the retracted position to the advanced position. This may be achieved by forming a pre-bent portion in the penetration tool 130 or making the penetration tool 130 from a shape memory material such as Nitinol.

The penetration tool 130 itself further comprises a guidewire lumen for accommodating a guidewire, which allows placement of the guidewire through the penetration tool 130.

FIG. 2 also illustrates how blood can flow past the inflated balloon 120 by flowing through the spiral channels 121.

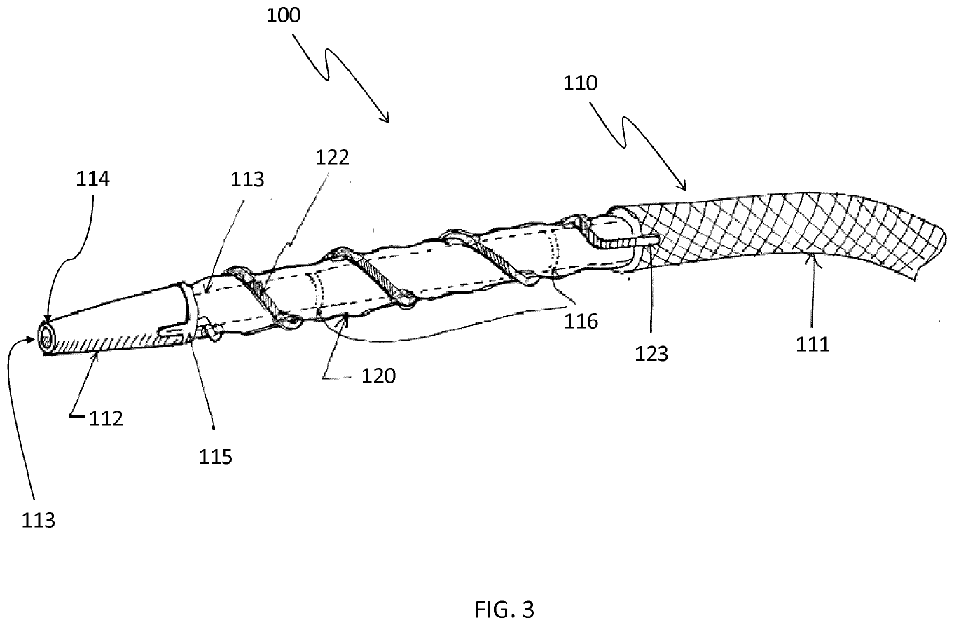

FIG. 3 shows the crossing catheter 110 with the inflatable balloon 120 in the deflated state. As can be seen in FIG. 3, the crossing catheter comprises a wire 122 which is wrapped in a spiral around the inflatable balloon 120. The wire 122 may be made from Nitinol or another metal such as steel. At the proximal end, the wire 122 is attached to the shaft 111 with a crimp 123. At the distal end, the wire 122 is attached to the radiopaque marker 115 and the distal tip 112. This allows the wire 122 to maintain its shape and stay in place when the balloon 120 is inflated, thereby forming the spiral channels 121 in the inflated balloon 120. The wire 122 has a rounded shape to prevent damaging the vessel wall and the balloon 120.

FIG. 3 also shows an outline of the penetration lumen 113 within the crossing catheter 110 in which the penetration tool 130 is disposed. The crossing catheter 110 may further comprise internal radiopaque marker bands 116 which allow the location of the inflatable balloon 120 to be easily determined under fluoroscopy. For example, the radiopaque marker bands 116 can be disposed at the distal and proximal ends of the balloon 120 to mark the location and length of the balloon 120. Alternatively, the radiopaque marker bands 116 can be used to mark a working surface of the balloon 120.

FIGS. 4A to 4G illustrate a method of using the catheter system 100 to form a fistula between an artery A and a vein V. The forming of a fistula is an essential step in a vascular bypass procedure or in a deep vein arterialisation procedure to treat peripheral artery disease.

Firstly, a suitable access site is formed, and a guidewire 140 is introduced into the artery A and advanced to the treatment site under fluoroscopy. The treatment site could, for example, be the site of a chronic total occlusion CTO in the artery where build-up of calcified plaque inside the artery A has caused a blockage in the vessel such that no or only very little blood can flow through the artery. An arteriovenous fistula can be formed to bypass this chronic total occlusion or formed as part of a deep vein arterialisation procedure.

The crossing catheter 110 together with the penetration tool 130 is then passed over the guidewire 140 such that the guidewire 140 is disposed within the guidewire lumen of the penetration tool 130, and introduced into the artery A through the access site. The crossing catheter 110 and penetration tool 130 are then advanced along the guidewire 140 by a physician to the treatment site where the fistula is to be formed. During advancement of the crossing catheter 110, the balloon 120 is in a deflated state.

FIG. 4A shows the crossing catheter at the treatment or cross-over site. The treatment site is usually heavily calcified and comprises plaque P in the artery wall resulting in an uneven lumen of the vessel. When the crossing catheter is at the treatment site, the physician partially advances the penetration tool 130 such that it partially extends from the distal tip 112 of the crossing catheter 120.

As shown in FIG. 4B, the physician then inserts a snare catheter 150 into the adjacent vein V through a different venous access site and advances the snare catheter 150 over a guidewire 153 to the venous side of the treatment site. The snare catheter 150 is advanced from an opposite side to the crossing catheter 110. The snare catheter 150 comprises a first expandable mesh 151 and a second expandable mesh 154 sequentially arranged. A radiopaque markers 152a is disposed at the distal end of the first expandable mesh 151, a second radiopaque marker 152b is disposed between the first expandable mesh 151 and the second expandable mesh 154, and a third radiopaque 152c marker is disposed at the proximal end of the second expandable mesh 154. The radiopaque markers 152 allow the first and second expandable mesh 151, 154 to be accurately located and positioned under fluoroscopy.

Once the snare catheter 150 is positioned on the venous side of the treatment site, the expandable mesh 151 is expanded from a contracted to an expanded configuration to stabilise the sharing catheter 150 in the vein V.

Meanwhile, the crossing catheter 110 in the artery A is rotated so that the distal end of the penetration tool 130 is facing the vein V. The guidewire 140 is then advanced through the guidewire lumen of the penetration tool 130 until it extends from the distal end of the penetration tool 130. The physician can then feel the wall of the artery using the guidewire 140 to determine a suitable crossing point for forming the fistula where the vessel wall is less calcified.

Next, as shown in the FIG. 4C, the inflatable balloon 120 is inflated through the inflation lumen in the crossing catheter 110. The inflated balloon 120 stabilises the crossing catheter 110 to allow the penetration tool 130 to penetrate the vessel walls between the artery A and vein to form the fistula. The spiral channels 121 in the balloon 120 provide better stabilisation in the heavily calcified and uneven lumen of the artery A. Furthermore, the spiral channel 121 allows blood to flow past the inflated balloon 120 during the fistula creation process and thereby reduces the risk of formation of a blood clot.

Once the balloon 120 is inflated, as shown in FIG. 4E, the physician then advances the penetration tool 130 such that it extends from the penetration port 114 of the crossing catheter 110. The sharp distal needle end of the penetration tool 130 deflects laterally and penetrates the vessel wall of the artery A and vein V to form a fistula F between the artery A and vein V.

FIG. 4F shows how, once the penetration tool 130 has reached the lumen of the vein V, the physician advances the guidewire 140 through the guidewire lumen in the penetration tool 130 into the vein V. The guidewire 140 is then advanced into contact with the expandable mesh 151 of the snare catheter 150, and the expandable mesh 151 is collapsed to snare the guidewire 140. Once the guidewire is secured to the expandable mesh 151, the snare catheter 150 with the guidewire 140 is pulled back through the vein V such that the guidewire 140 is positioned in the vein V.

The inflatable balloon 120 is then deflated and the crossing catheter 110 is translated over the guidewire 140 into the fistula F. The tapered distal tip 112 helps to widen the fistula F and allow the crossing catheter 110 to pass into the fistula F. Once the balloon 120 is positioned within the fistula F, the balloon 120 is inflated to expand the size of the fistula F, as shown in FIG. 4G. This avoids the need for a separate balloon catheter to be introduced to enlarge the fistula and significantly simplifies and shortens the procedure.

The crossing catheter 110 and snare catheter 150 can then be removed from the patient's body. A stent can be inserted into the fistula F to stabilise it. If a vascular bypass procedure is performed, a second fistula can be formed in the same manner on the other side of the chronic total occlusion CTO and a stent graft can be inserted to bypass the chronic total occlusion CTO.

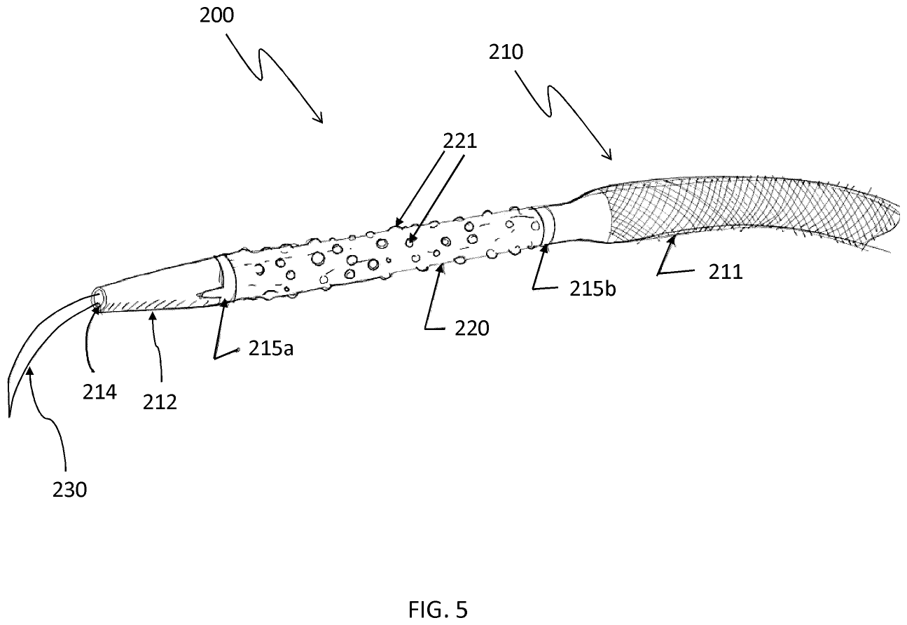

FIG. 5 shows an alternative embodiment of a catheter system 200 comprising a crossing catheter 210 and a penetration tool 230. The penetration tool 230 is identical to penetration tool 130 and located within the penetration lumen 213 of the crossing catheter 210.

The crossing catheter 210 is also similar to crossing catheter 110 in that it comprises a shaft 211 connected to an inflatable balloon 220 which is connected to a distal tip 212 having a distal penetration port 214. The inflatable balloon is in the deflated state in FIG. 5. The crossing catheter 210 further comprises a first radiopaque marker 215a and a second radiopaque marker 215b which mark the ends of the inflatable balloon 220 and allow the inflatable balloon 220 and distal tip 212 to be identified and located under fluoroscopy.

The crossing catheter 210 differs from the crossing catheter 110 of FIGS. 1 to 4 in that the inflatable balloon 220 comprises a different type of blood bypass element. Rather than wire 122 and spiral channels 121, inflatable balloon 220 comprises a number of small bumps or nodules 221 on the surface of the balloon 220. The bumps or nodules 221 extend substantially along the entire length of the balloon 220. This means that the bumps 221 may extend along 60% to 100%, preferably 80% to 100%, of the entire length of the balloon 220. The bumps 221 may be formed from the same material as the balloon 220. For example, droplets of nylon may be deposited on the surface of the balloon 220 to form the bumps 221.

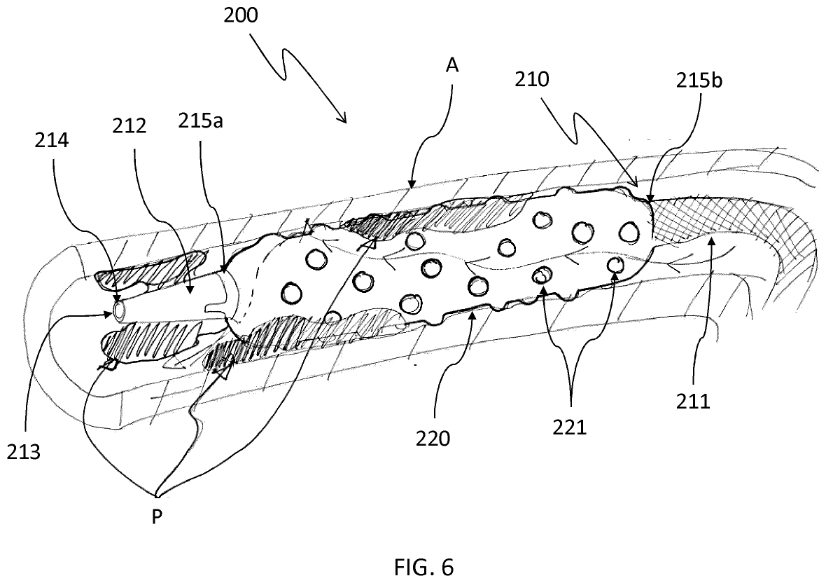

FIG. 6 shows the same crossing catheter 210 positioned in an artery A with the inflatable balloon 220 in the inflated state. As can be seen the bumps 221 of the inflated balloon 220 engage the lumen of the artery A. The bumps 221 provide engagement points for a heavily calcified and uneven lumen comprising plaque deposits P and therefore, similar to inflatable balloon 120, the inflatable balloon 220 provides better stabilisation of the crossing catheter 210 in an uneven and heavily calcified lumen. Furthermore, the raised bumps 221 provide for passages between the bumps 221 through which blood can flow, even when the balloon 220 is inflated. Similar to balloon 120, balloon 220 therefore allows blood flow through the vessel during the fistula forming process and reduces the risk of blood clot formation.

Moreover, the balloon 220 reduces the surface area which is in contact with the lumen wall, as the raised bumps 221 act as contact points with the lumen wall. This means that the risk of trauma to the lumen wall is reduced. Deep Vein Arterialization procedures have a long procedural time and reducing the time that any component is in contact with the vessel wall can improve the chances of success of the procedure.

To that extent, the balloon 220 may further comprise a tapered shape. For example, in the inflated state, the diameter of the balloon 220 may decrease from the proximal end and the distal end towards a centre portion. This further reduces the surface area which is in contact with the lumen wall and also allows better blood flow past the inflated balloon 220.

The catheter system 200 including crossing catheter 210 and penetration tool 230 may be used in exactly the same way as catheter system 100 described above in relation to FIGS. 4A-G.

Various modifications will be apparent to those skilled in the art.

The inflatable balloon 120 may comprise multiple blood bypass elements, such as two or more spiral channels 121.

The blood bypass element is not limited to a spiral channel 121 or bumps but may take different forms such as spiral ridges, straight channels or straight ridges.

The inflatable balloon 120 may comprise a combination of different blood bypass elements such as, for example, spiral channels and spiral ridges, spiral channels and bumps, or straight channels and straight ridges.

The inflatable balloon 120 may also have a tapered shape wherein, in the inflated state, the diameter of the balloon decreases from the proximal end and the distal end towards the centre portion.

The penetration port 114 may not be disposed at the distal end of the crossing catheter 110, but may be disposed proximally or distally adjacent the inflatable balloon 120.

The penetration port 114 may also be a lateral opening in the crossing catheter 110. This lateral opening may be disposed at the distal end of the crossing catheter 110, or proximally or distally adjacent the inflatable balloon 120.

The crossing catheter 110 may comprise a second inflatable balloon which is positioned proximal of inflatable balloon 120.

The second inflatable balloon may also comprise one, two or more blood bypass elements.

The blood bypass elements of the second inflatable balloon may be the same or different from those of inflatable balloon 120 and may be, for example, spiral channels, spiral ridges, straight channels, straight ridges or bumps.

The penetration port 114 may be disposed between the first and second inflatable balloon.

The crossing catheter 110 may comprise a second guidewire lumen for a second guidewire which is separate from the guidewire lumen of the penetration tool 130. This guidewire may be used to guide the crossing catheter to the treatment site.

The crossing catheter 110 may not comprise the radiopaque marker 115 or radiopaque marker bands 116.

The crossing catheter 110 may also comprise two radiopaque markers 115a, 115b similar to markers 215a, 215b, which mark the distal and proximal end of the inflatable balloon 120.

The penetration tool 130 may not bend to deflect laterally when it is moved from the retracted position to the advanced position. Rather, for example, the penetration port 114 may be disposed at an angle to allow the penetration tool to exit the crossing catheter at an angle.

The snare catheter 150 may only comprise one expandable mesh.

The snare catheter may not comprise any radiopaque markers 152.

All of the above are fully within the scope of the present disclosure and are considered to form the basis for alternative embodiments in which one or more combinations of the above described features are applied, without limitation to the specific combination disclosed above.

In light of this, there will be many alternatives which implement the teaching of the present disclosure. It is expected that one skilled in the art will be able to modify and adapt the above disclosure to suit its own circumstances and requirements within the scope of the present disclosure, while retaining some or all technical effects of the same, either disclosed or derivable from the above, in light of his common general knowledge in this art. All such equivalents, modifications or adaptations fall within the scope of the present disclosure.

1. A catheter system for forming a fistula between two adjacent blood vessels, the catheter system comprising:
a crossing catheter for introduction into a blood vessel, the crossing catheter having a distal end section and an inflatable balloon disposed in the distal end section for stabilizing the crossing catheter in the blood vessel;
a penetration tool for penetrating vessel walls of two adjacent blood vessels to form the fistula, wherein the penetration tool is disposed within the crossing catheter and is movable between a retracted position and an advanced position,
wherein, in the inflated state, the inflatable balloon comprises at least one blood bypass structure for allowing blood to flow past the inflated balloon,
wherein the crossing catheter has a penetration lumen for accommodating the penetration tool and a distal end of the penetration lumen ends in a penetration port through which the penetration tool can exit the crossing catheter, and wherein the penetration port is disposed at the distal end of the crossing catheter.

2. The catheter system of claim 1, wherein the blood bypass structure comprises a spiral channel on an outer surface of the balloon.

3. The catheter system of claim 2, wherein the spiral channel is formed by a wire wrapped in a spiral around the outer surface of the balloon.

4. (canceled)

5. The catheter system of claim 1, wherein the blood bypass structure comprises a spiral ridge on an outer surface of the balloon.

6. (canceled)

7. The catheter system of claim 1, wherein the blood bypass structure comprises multiple bumps on the surface of the inflatable balloon.

8-13. (canceled)

14. The catheter system of claim 1, wherein the penetration port is disposed distally adjacent the inflatable balloon.

15. (canceled)

16. The catheter system of claim 1, wherein the penetration port comprises a longitudinal or lateral opening in the crossing catheter.

17-18. (canceled)

19. The catheter system of claim 1, wherein a distal end of the penetration tool deflects laterally as the penetration tool is moved distally from the retracted position to the advanced position.

20. (canceled)

21. The catheter system of claim 1, wherein the penetration tool comprises a guidewire lumen for accommodating a guidewire, wherein the guidewire lumen allows placement of the guidewire through the fistula formed by the penetration tool.

22. The catheter system of claim 21, wherein the crossing catheter comprises a second guidewire lumen for accommodating a second guidewire.

23. The catheter system of claim 1, wherein the inflatable balloon is a first inflatable balloon, the crossing catheter further comprises a second inflatable balloon, wherein the second inflatable balloon, in the inflated state, comprises at least one blood bypass structure for allowing blood to flow past the inflated balloon.

24-25. (canceled)

26. The catheter system of claim 1, wherein the inflatable balloon is at least partly made from a woven or spun thread.

27. A catheter system for forming a fistula between two adjacent blood vessels, the catheter system comprising:
a crossing catheter for introduction into a blood vessel, the crossing catheter having a distal end section and an inflatable balloon disposed in the distal end section for stabilizing the crossing catheter in the blood vessel;
a needle for penetrating vessel walls of two adjacent blood vessels to form the fistula, wherein the needle is disposed within the crossing catheter and is movable between a retracted position and an advanced position,
wherein the inflatable balloon is adapted for allowing blood to flow past the inflated balloon,
wherein the crossing catheter has a penetration lumen for accommodating the needle and a distal end of the penetration lumen ends in a penetration port through which the needle can exit the crossing catheter, and wherein the penetration port is disposed at the distal end of the crossing catheter.

28. The catheter system of claim 27, wherein the inflatable balloon comprises a spiral channel on an outer surface of the balloon for allowing blood to flow past the inflated balloon.

29. The catheter system of claim 28, wherein the spiral channel is formed by a wire wrapped in a spiral around the outer surface of the balloon.

30. (canceled)

31. The catheter system of claim 27, wherein the inflatable balloon comprises a spiral ridge on an outer surface of the balloon for allowing blood to flow past the inflated balloon.

32. (canceled)

33. The catheter system of claim 27, wherein the inflatable balloon comprises multiple bumps on the outer surface of the balloon.

34-38. (canceled)

39. The catheter system of claim 37, wherein the penetration port comprises a lateral or longitudinal opening in the crossing catheter.

40. The catheter system of claim 27, wherein a distal end of the needle deflects laterally as the needle is moved distally from the retracted position to the advanced position.

41. A method of forming a fistula between two adjacent blood vessels using a catheter system comprising a crossing catheter having a distal end section and an inflatable balloon disposed in the distal end section, and a penetration tool, the method comprising:
introducing the crossing catheter into a blood vessel;
advancing the distal end of the crossing catheter to a predetermined point where the fistula is to be formed;
stabilizing the crossing catheter by inflating the inflatable balloon, wherein the inflatable balloon comprises at least one blood bypass structure for allowing blood to flow past the inflated balloon;
moving the penetration tool from a retracted position and an advanced position to form the fistula.

42. The catheter of claim 1, wherein the inflatable balloon is configured for expanding the size of the fistula when inflated while positioned within the fistula.
